(19) United States
(12) Patent Application Publication
(10) Pub. No.: US 2009/0281494 A1
(43) Pub. Date: Nov. 12, 2009

(54) Heat Sterilizable Ambulatory Infusion Devices

(76) Inventor: Stephen D. Das, The Woodlands, TX (US)

Correspondence Address: HENRICKS SLAVIN AND HOLMES, LLP;ADVANCED BIONICS CORPORATION, 840 APOLLO STREET, SUITE 200, EL SEGUNDO, CA, 90245, US

(21) Appl. No.: 12/116,181

(22) Filed: May 6, 2008

(51) Int. Cl. A61M 5/152

(52) U.S. Cl. 604/132

(57) ABSTRACT

Ambulatory infusion devices with components that may be heat sterilized.

### BACKGROUND OF THE INVENTIONS

1. Field of Inventions

The present inventions relate generally to ambulatory infusion devices.

2. Description of the Related Art

Ambulatory infusion devices, such as implantable infusion devices and externally carried infusion devices, have been used to provide a patient with a medication or other substance (collectively “infusible substance”) and frequently include a reservoir and a fluid transfer device. The reservoir is used to store the infusible substance and, in some instances, implantable infusion devices are provided with a fill port that allows the reservoir to be transcutaneously filled (and/or re-filled) with a hypodermic needle. The reservoir is coupled to the fluid transfer device, which is in turn connected to an outlet port. A catheter, which has at least one outlet at the target body region, may be connected to the outlet port. As such, infusible substance may be transferred from the reservoir to the target body region(s) by way of the fluid transfer device and catheter.

Ambulatory infusion devices are sterilized prior to being implanted or otherwise associated with a patient. Such sterilization is often part of the manufacturing and/or packaging processes. Although many medical devices can be quickly and inexpensively heat sterilized (e.g. steam sterilized at a temperature of 121-132° C. for up to three hours, or dry heat sterilized at a temperature of 160-170° C. for up to two and one-half hours), conventional ambulatory infusion devices that include piezoelectric components have been heretofore sterilized by far more time consuming and costly procedures because the piezoelectric components employed in conventional ambulatory infusion devices have poor heat durability and may not function properly after heat sterilization. One such sterilization procedure is a three-step procedure that involves a first ethylene oxide sterilization (“EtO sterilization”), a “sterile fluid fill,” and a second EtO sterilization. In the first EtO sterilization, ethylene oxide is pumped through the internal fluid path of the ambulatory infusion device, i.e., through the inlet, the reservoir, the pump, the outlet and the fluidic connections therebetween. EtO sterilization, which costs approximately $5-10 per ambulatory infusion device, typically takes a minimum of ten days and, if the device has to be shipped to another location, another ten days may be added to the EtO sterilization process. The “sterile fluid fill” involves using a relatively complex apparatus to fill the ambulatory infusion device with water that has a pharmacological level of sterility and typically costs approximately $20. The second EtO sterilization, which sterilizes the exterior of the ambulatory infusion devices involves placing the device in a pouch and filling the pouch with ethylene oxide. Here too, the EtO sterilization may take up to 10-20 days and cost approximately $5-10.

### SUMMARY OF THE INVENTIONS

Ambulatory infusion devices in accordance with at least some of the present inventions include piezoelectric components such as, for example, a sound generation device and/or a fluid transfer device actuator, that are configured to retain their piezoelectric characteristics after being exposed to a heat sterilization process. Such ambulatory infusion devices substantially reduce the time and expense associated with the manufacture and/or packaging of ambulatory infusion devices because they do not require the expensive and time consuming sterilization procedures associated with conventional ambulatory infusion devices.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 is a plan view of an implantable infusion device in accordance with one embodiment of a present invention.

FIG. 2 is a plan view of the implantable infusion device illustrated in FIG. 1 with the cover removed.

FIG. 3 is a partial section view taken along line 3-3 in FIG. 1.

FIG. 4 is a block diagram of the implantable infusion device illustrated in FIGS. 1-3.

FIG. 5 is a plan view of the interior side of the implantable infusion device cover illustrated in FIG. 1.

FIG. 6 is a plan view of an implantable infusion device in accordance with one embodiment of a present invention.

FIG. 7 is a block diagram of the implantable infusion device illustrated in FIG. 6.

FIG. 8 is a partial section view of a pair of fluid transfer devices with a common actuator in accordance with one embodiment of a present invention.

FIG. 9 is a section view of a fluid transfer device in accordance with one embodiment of a present invention.

FIG. 10 is a plan view of an implantable infusion device in accordance with one embodiment of a present invention.

FIG. 11 is a plan view of an implantable infusion device in accordance with one embodiment of a present invention.

FIG. 12 is a plan view of an implantable infusion device in accordance with one embodiment of a present invention.

FIG. 13 is a schematic view of the implantable infusion device illustrated in FIG. 12.

FIG. 14 is a partial section view of a fluid transfer device valve in accordance with one embodiment of a present invention.

FIG. 15 is a partial section view of a fluid transfer device in accordance with one embodiment of a present invention.

FIG. 16 is a flow chart in accordance with one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. The present inventions are also not limited to the exemplary implantable infusion devices described herein and, instead, are applicable to other implantable or otherwise ambulatory infusion devices that currently exist or are yet to be developed.

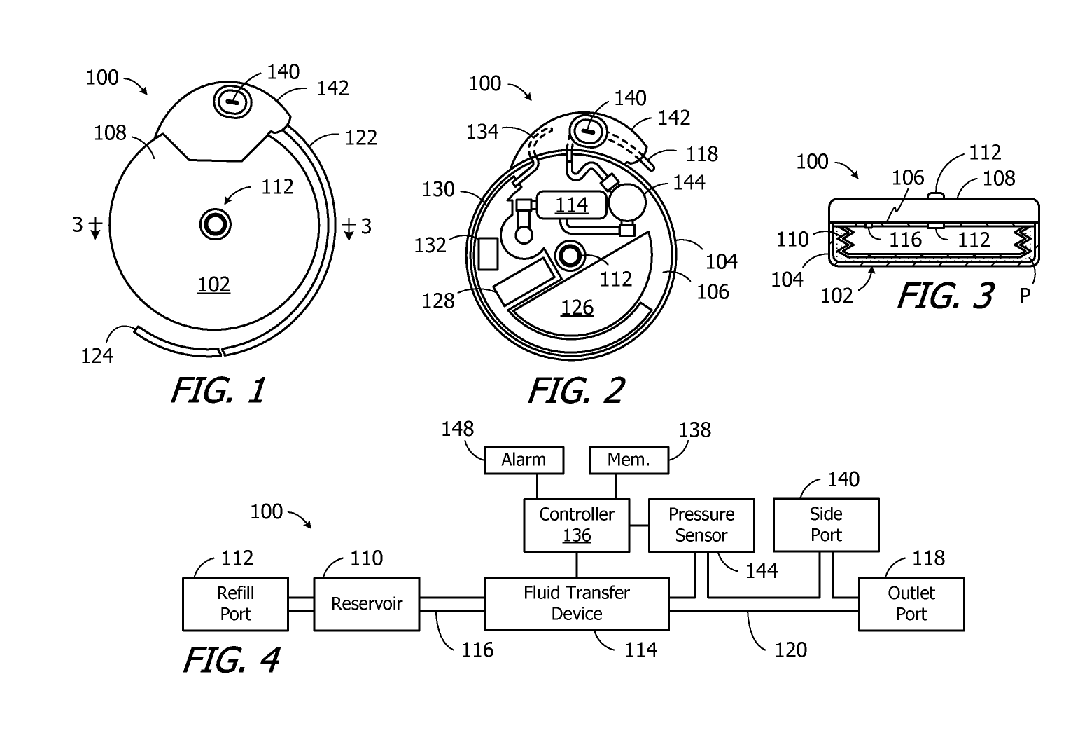

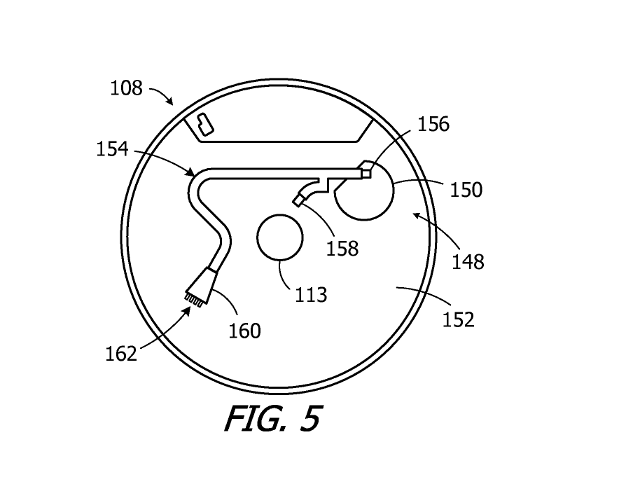

One example of an implantable infusion device in accordance with a present invention is generally represented by reference numeral 100 in FIGS. 1-4. As used herein, an “implantable infusion device” is a device that includes a reservoir and an outlet, and is sized, shaped and otherwise constructed (e.g. sealed) such that both the reservoir and outlet can be simultaneously carried within the patient's body. The exemplary infusion device 100 includes a housing 102 (e.g. a titanium housing) with a bottom portion 104, an internal wall 106, and a cover 108. An infusible substance (e.g. medication) may be stored in a reservoir 110 that is located within the housing bottom portion 104. The reservoir 110 may be replenished by way of a fill port 112 that extends from the reservoir, through the internal wall 106, to the exterior of the cover 108. The cover includes an aperture 113 (FIG. 5) to accommodate the fill port 112. A hypodermic needle (not shown), which is configured to be pushed through the fill port 112, may be used to replenish the reservoir 110.

A wide variety of reservoirs may be employed. In the illustrated embodiment, the reservoir 110 is in the form of a titanium bellows that is positioned within a sealed volume defined by the housing bottom portion 104 and internal wall 106. The remainder of the sealed volume is occupied by propellant P, which may be used to exert negative pressure on the reservoir 110. Other reservoirs that may be employed in the present infusion devices include reservoirs in which propellant exerts a positive pressure. Still other exemplary reservoirs include negative pressure reservoirs that employ a movable wall that is exposed to ambient pressure and is configured to exert a force that produces an interior pressure that is always negative with respect to the ambient pressure.

The exemplary ambulatory infusion device 100 illustrated in FIGS. 1-4 also includes a fluid transfer device 114. The inlet of a fluid transfer device 114 is coupled to the interior of the reservoir 110 by a passageway 116, while the outlet of the fluid transfer device is coupled to an outlet port 118 by a passageway 120. Operation of the fluid transfer device 114 causes infusible substance to move from the reservoir 110 to the outlet port 118. A catheter 122 may be connected to the outlet port 118 so that the infusible substance passing through the outlet port will be delivered to a target body region in spaced relation to the infusion device 100 by way of the outlet(s) 124 at the end of the catheter.

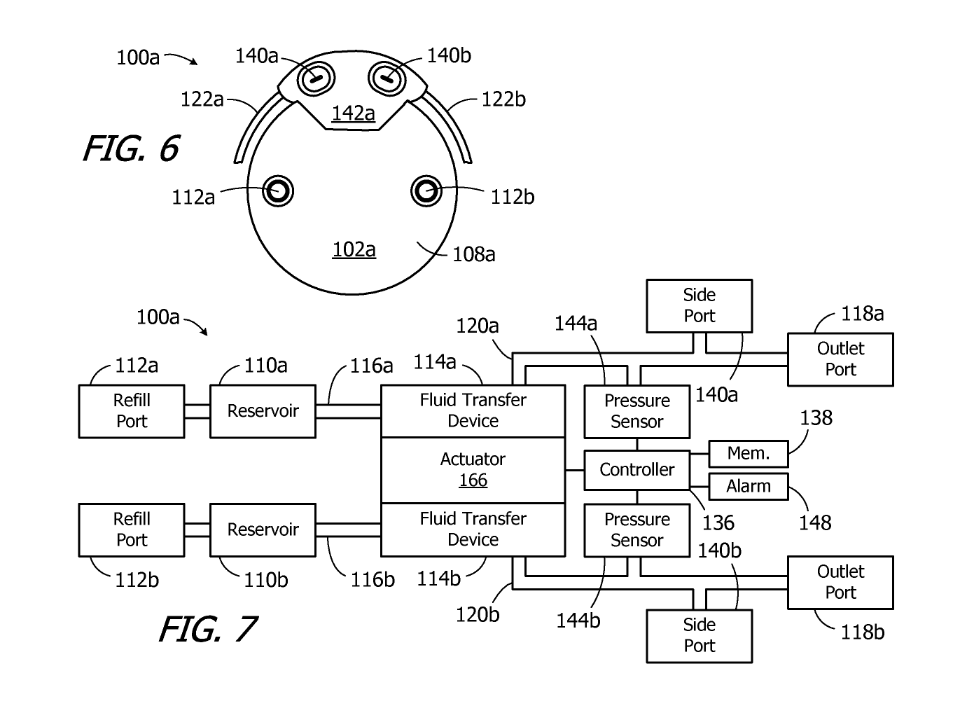

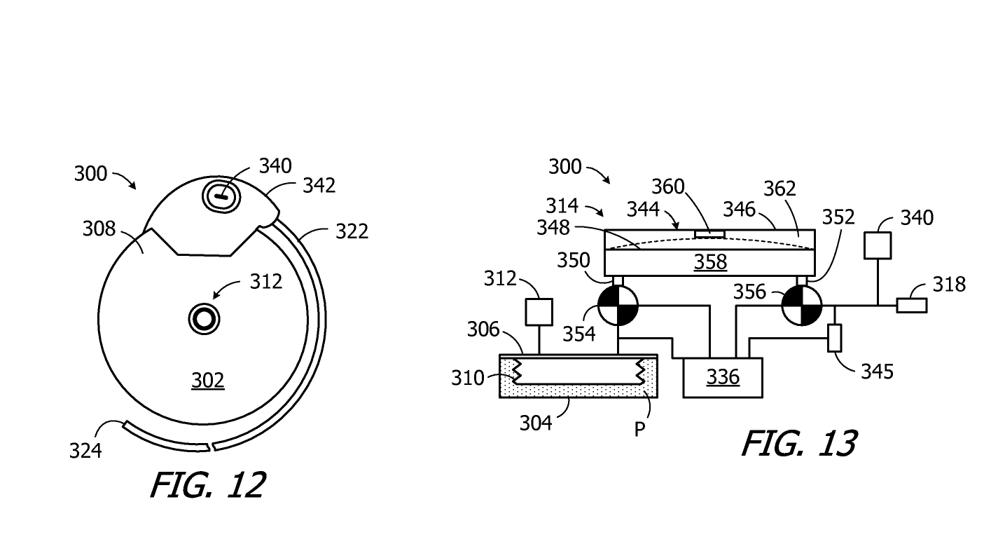

A wide variety of fluid transfer devices may be employed. In the illustrated embodiment, the fluid transfer device 114 is in the form of an electromagnet pump. The present inventions are not, however, limited to electromagnet pumps and may include other types of fluid transfer devices. Such devices include, but are not limited to, other electromagnetic pumps, solenoid pumps, piezoelectric pumps (discussed below with reference to FIGS. 6-9), accumulator and valve arrangements (discussed below with reference to FIGS. 12-14) and any other mechanical or electromechanical pulsatile pump. In the exemplary context of implantable drug delivery devices, and although the volume/stroke magnitude may be increased in certain situations, the fluid transfer devices will typically deliver about 1 microliter/stroke, but may be more or less (e.g. about 0.25 microliter/stroke or less) depending on the particular fluid transfer device employed. Additionally, although the exemplary fluid transfer device 114 is provided with internal valves (e.g. a main check valve and a bypass valve), valves may also be provided as separate structural elements that are positioned upstream of and/or downstream from the associated fluid transfer device.

Energy for the fluid transfer device 114, as well for other aspects of the exemplary infusion device 100, is provided by the battery 126 illustrated in FIG. 2. In the specific case of the fluid transfer device 114, the battery 126 is used to charge one or more capacitors 128, and is not directly connected to the fluid transfer device itself. The capacitor(s) 128 are connected to an electromagnet coil in the fluid transfer device 114, and disconnected from the battery 126, when the electromagnet coil is being energized, and are disconnected from the electromagnet coil and connected to the battery when the capacitor(s) are being recharged and/or when the fluid transfer device is at rest. The capacitor(s) 128 are carried on a board 130. A communication device 132, which is connected to an antenna 134, is carried on the same side of the board 130 as the capacitor(s) 128. The exemplary communication device 132 is an RF communication device. Other suitable communication devices include, but are not limited to, oscillating magnetic field communication devices, static magnetic field communication devices, optical communication devices, ultrasound communication devices and direct electrical communication devices.

A controller 136 (FIG. 4), such as a microprocessor, microcontroller or other control circuitry, is carried on the other side of the board 130. The controller controls the operations of the infusion device 100 in accordance with instructions stored in memory 138 and/or provided by an external device (e.g. a remote control) by way of the communication device 132. For example, the controller 136 may be used to control the fluid transfer device 114 to supply fluid to the patient in accordance with, for example, a stored basal delivery schedule or a bolus delivery request. The controller 136 may also be used to monitor sensed pressure and perform the analytical functions described below.

Referring to FIGS. 1, 2 and 4, the exemplary infusion device 100 is also provided with a side port 140 that is connected to the passageway 120 between the outlet of the fluid transfer device 114 and the outlet port 118. The side port 140 facilitates access to an implanted catheter 122, typically by way of a hypodermic needle. For example, the side port 140 allows clinicians to push fluid into the catheter 122 and/or draw fluid from the catheter for purposes such as checking catheter patency, sampling CSF, injecting contrast dye into the patient and/or catheter, removing medication from the catheter prior to dye injection, injecting additional medication into the region at the catheter outlet 124, and/or removing pharmaceuticals or other fluids that are causing an allergic or otherwise undesirable biologic reaction.

The outlet port 118, a portion of the passageway 120, the antenna 134 and the side port 140 are carried by a header assembly 142. The header assembly 142 is a molded, plastic structure that is secured to the housing 102. The housing 102 includes a small aperture through which portions of the passageway 120 are connected to one another, and a small aperture through which the antenna 134 is connected to the board 130.

The exemplary infusion device 100 illustrated in FIGS. 1-4 also includes a pressure sensor 144 that is connected to the passageway 120 between the outlet of the fluid transfer device 114 and the outlet port 118. As such, the pressure sensor 144 senses the pressure at the outlet port 118 which, in the illustrated embodiment, is also the pressure within the catheter 122. The pressure sensor 144 is connected to the controller 136 and may be used to analyze a variety of aspects of the operation of the exemplary implantable infusion device 100. For example, pressure measurements may be used by the controller 136 to determine whether or not there is a blockage in the catheter 122. The controller 136 may perform a variety of different functions in response to a determination that the fluid transfer device 114 is not functioning properly or a determination that the catheter 122 is blocked. For example, the controller 136 may actuate a sound generator 148 (or other alarm) that is located within the housing 102 in order to signal that the fluid transfer device 114 is not functioning properly or the catheter 122 is blocked.

Turning to FIG. 5, the sound generator 148 may be a piezoelectric sound generator and, in the illustrated embodiment, the sound generator includes a piezoelectric actuator 150 and the housing cover 108. The piezoelectric actuator 150 is secured to the inner surface 152 of the housing cover 108, either directly or with a brass disk (not shown) between piezoelectric actuator and the inner surface, through the use of an adhesive or other suitable instrumentality. A flex cable 154, with exposed metal (e.g. copper) traces 156 and 158 on one end and a connector 160 with a plurality of metal (e.g. nickel) tabs 162 on the other, may be used to connect the piezoelectric actuator 150 to an AC voltage supply (not shown) on the board 130. The flex cable 154 includes a first wire (not shown), which is connected to the copper trace 158 and two of the tabs 162, and a second wire (not shown), which is connected to the copper trace 160 and the other two tabs 162. The piezoelectric actuator 150 will repeatedly shrink and expand as AC voltage is applied across piezoelectric actuator which, in turn, causes the housing cover 108 to flex inwardly and outwardly and produce sound waves.

The piezoelectric material used to form the piezoelectric actuator 150 is a heat sterilizable piezoelectric material. As used herein, a “heat sterilizable piezoelectric material” is a piezoelectric material (e.g. a piezoelectric ceramic) that may be heated to temperatures associated with heat sterilization without having its crystalline structure altered to such an extent that, even after cooling to room temperature (e.g. about 20° C.), it no longer possesses piezoelectric characteristics. The temperature above which the crystalline structure changes from piezoelectric to non-piezoelectric, and all piezoelectric properties are lost, is referred to as the Curie temperature (Tc) or Curie point. Heat sterilization takes place at temperatures above 105° C. and, accordingly, heat sterilizable piezoelectric materials are piezoelectric materials with a Tc of at least 105° C. In some embodiments, the heat sterilizable piezoelectric materials will have a Tc of 120-170° C. or more. In some embodiments, the heat sterilizable piezoelectric materials will have a Tc of up to 360° C., or more.

It should be noted that heating time may also effect piezoelectric material. At least some piezoelectric materials that are heated to a temperature which is somewhat close to, but below, the Tc may experience some degradation of their piezoelectric characteristics if the heating is of a sufficient duration. Accordingly, in some instances, it may be desirable to select piezoelectric materials whose Tc's are significantly higher than the temperatures to which the materials may be exposed during sterilization. For example, it may be desirable to select piezoelectric materials, whose Tc's are approximately twice the temperature to which the materials may be exposed during sterilization, for use as heat sterilizable piezoelectric materials.

By way of example, but not limitation, suitable heat sterilizable piezoelectric materials include various piezoelectric ceramics, such as certain types of PbZrTiO₃ (“PZT”), that have a Tc of at least 105° C. Suitable heat sterilizable piezoelectric materials may also include various piezoelectric ceramics, such as certain types of PZT, that have a Tc of at least about twice the temperature to which the material may be exposed during sterilization. For example, piezoelectric materials with Tc's of at least 242-264° C. may be used if steam sterilization at temperatures of 121-132° C. for up to three hours is to be employed, while piezoelectric materials with Tc's of at least 320-340° C. may be used if dry heat sterilization at temperatures of 160-170° C. for up to two and one-half hours is a possibility. Specific examples of suitable heat sterilizable piezoelectric ceramics include, for example, the piezoelectric ceramics disclosed in U.S. Pat. No. 5,645,753 and U.S. Patent Pub. No. 2007/0247028, which are incorporated herein by reference, as well as the material identified as K-350 (Tc=360° C.) and sold by Piezo Technologies (formerly Keramos), which is located in Indianapolis, Ind.

It should be noted that the use of heat sterilizable piezoelectric materials in an ambulatory infusion device is not limited to sound generators. Piezoelectric materials may also be associated with fluid transfer devices and other components. For example, an ambulatory infusion device may include a pair of fluid transfer devices that share a common piezoelectric actuator. One example of such a device is the implantable infusion device generally represented by reference numeral 100a in FIGS. 6 and 7. The implantable infusion device 100a is substantially similar to the implantable infusion device 100 described above with reference to FIGS. 1-4 and similar elements are represented by similar reference numerals. The implantable infusion device 100a is, however, configured to deliver fluid to the patient by way of two different outlets and, if so desired, deliver fluid from the two different outlets at two different rates.

To that end, the implantable infusion device 100a includes two of some of the elements found in the implantable infusion device 100, and only one of other elements. For example, the implantable infusion device 100a includes a housing 102a (e.g. a titanium housing), a pair of reservoirs 110a and 110b within the housing, a pair of fill ports 112a and 112b that respectively extend from the reservoirs 110a and 110b to the exterior of the cover 108a, and a pair of fluid transfer devices 114a and 114b with a common actuator 166 (discussed below). The inlets of the fluid transfer devices 114a and 114b are coupled to the interiors of the reservoirs 110a and 110b by passageways 116a and 116b, while the outlets of the fluid transfer devices are coupled to outlet ports 118a and 118b by passageways 120a and 120b. Catheters 122a and 122b may be connected to the outlet ports 118a and 118b. The exemplary implantable infusion device 100a also includes a controller 136 and memory 138, side ports 140a and 140b, which are carried in a header assembly 142a, pressure sensors 144a and 144b, and a sound generator 148. The sound generator 148 may be a piezoelectric sound generator that includes heat sterilizable piezoelectric material, or some other type of alarm that is capable of withstanding sterilization temperatures.

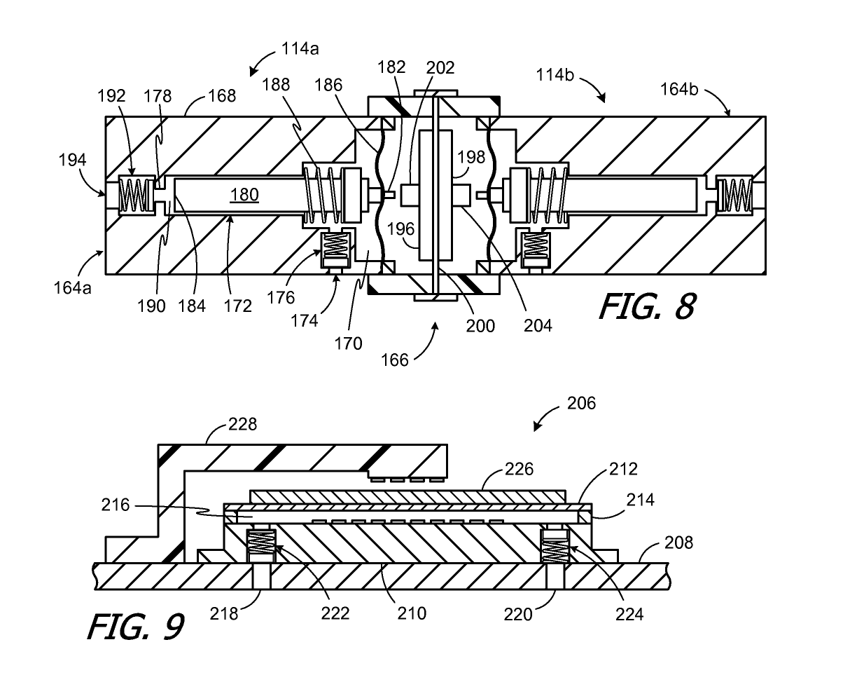

Turning to FIG. 8, the exemplary fluid transfer devices 114a and 114b each include a pump head 164a and 164b and share the common actuator 166. Although the present inventions are not so limited, the pump head 164a and 164b in the illustrated embodiment are identical and each pump head has a housing 168 with an inlet chamber 170 that leads to a piston channel 172. Fluid from the associated reservoir enters the piston channel 172 by way of an inlet 174 and a check valve 176 and exits the piston channel by way of a channel outlet port 178. A piston 180 is mounted in the channel 172 for reciprocal linear movement between an intake position (shown) and a discharge position. The piston 180, which has a strike pin 182 at one end and a piston face 184 at the other end, is retained by a spring diaphragm 186 that seals the inlet chamber 170. A return spring 188 biases the piston away from the channel outlet port 178. A pump chamber 190 is defined between the piston face 184 and the channel outlet port 178, and a check valve 192 is mounted between channel outlet port and a pump head outlet 194.

The common actuator 166 is piezoelectric actuator that can be actuated in more than one actuation mode to independently drive (or not drive) each of the pump heads 164a and 164b. The actuator 166 is in its neutral mode orientation in FIG. 8 and includes first and second piezoceramic disks 196 and 198 that are carried by a flexible diaphragm 200. The piezoceramic disks 196 and 198 carry hammers 202 and 204. In the first actuation mode, a voltage is applied across the piezoceramic disk 196, thereby causing the disk to bend in the first direction (i.e., to the left in FIG. 8). The hammer 202 will, in turn, strike the strike pin 182 of the pump head 164a and drive the piston 180 to the discharge position. The piezoceramic disk 196, as well the remainder of the common actuator 166, will return to the neutral mode illustrated in FIG. 8 when the voltage is removed. Similarly, in the second actuation mode, a voltage is applied across the piezoceramic disk 198, thereby causing the disk to bend in the second direction (i.e., to the right in FIG. 8). The hammer 204 will, in turn, strike the strike pin of the pump head 164b and drive the associated piston to the discharge position.

With respect to materials, the exemplary piezoceramic disks 196 and 198 may be formed from any suitable heat sterilizable piezoelectric ceramic including, but not limited to, the heat sterilizable piezoelectric ceramics described above.

Other common actuators formed from heat sterilizable piezoelectric materials may also be employed. By way of example, a single piezoceramic disk that bends in opposite directions based on the polarity of the applied voltage, and has an unbent neutral state, may be carried on the flexible diaphragm 200 in place of the disks illustrated in FIG. 8. Cantilevered piezoelectric elements, which are another alternative, eliminate the need for the flexible diaphragm. Additionally, in any piezoelectric common actuator, the hammer(s) may be eliminated so that a piezoceramic element strikes the strike pin 202 or the corresponding portion of some other fluid transfer device.

Additional details concerning infusion devices with multiple reservoirs, multiple fluid transfer devices and piezoelectric actuators may be found in U.S. Patent Pub. No. 2006/0270983, which is incorporated herein by reference.

Another exemplary fluid transfer device that may employ a piezoelectric actuator is the micro-diaphragm fluid transfer device generally represented by reference numeral 206 in FIG. 9. The fluid transfer device 206 includes a base plate 208, a valve plate 210, and a diaphragm 212 that is mounted on the valve plate with a spacer ring 214 such that a pump chamber 216 is defined between the valve plate and diaphragm. The base plate 208 includes an inlet 218 and an outlet 220 which are connected to the pump chamber 216 by way of an inlet valve 222 and an outlet valve 224 that are located within the valve plate 210. The exemplary pump 206 also includes a piezoelectric actuator 226 that is used to deform the diaphragm 212 and, accordingly, increase and decrease the volume of the pump chamber 216. With respect to materials, the exemplary piezoelectric actuator 226 may be formed from any suitable heat sterilizable piezoelectric material including, but not limited to, the heat sterilizable piezoelectric materials described above.

In the illustrated embodiment, the diaphragm 212 bottoms out on the valve plate 210 during the pump stroke, while a stop arm 228 may limit the travel in the return stroke direction. Both the valve plate 210 and the stop arm 228 may be textured to prevent adhesion thereto.

Additional details concerning the fluid transfer device illustrated in FIG. 9 may be found in U.S. Patent Pub. No. 2007/0128055, which is incorporated herein by reference.

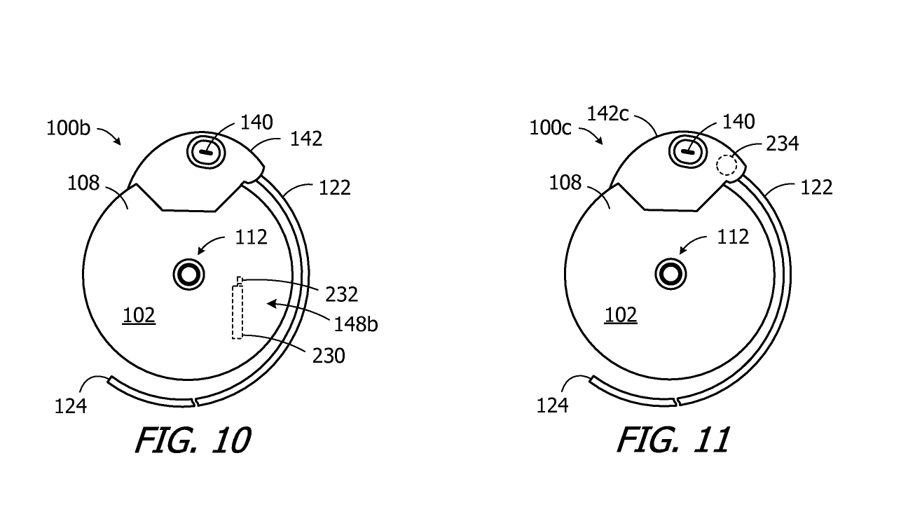

It should be noted here that ambulatory medical devices in accordance with the present inventions may include vibrating devices that are capable of withstanding the temperatures associated with heat sterilization processes. For example, instead of the sound generator 148, ambulatory medical devices may include a vibrator alarm (e.g. a micro electric motor with an imbalanced load on the motor spindle) to indicate when, among other things, the fluid transfer device is not functioning properly or the catheter is blocked. The implantable infusion device 100b illustrated in FIG. 10 is substantially similar to the implantable infusion device 100 described above with reference to FIGS. 1-4 and similar elements are represented by similar reference numerals. The implantable infusion device 100b includes an internal vibration generator 148b with a micro electric motor 230 (or “actuator”) and an imbalanced load 232 carried on the motor spindle. The vibration generator 148b may be mounted on the cover 108 or any other suitable structure. In some embodiments, the implantable infusion device 100b may also include the sound generator 148 described above with reference to FIG. 5.

Ambulatory medical devices may also include ultrasonic vibration devices that are used to break up encapsulations and other deposits at or near the catheter outlet(s). One example of such a medical device is the implantable infusion device 100c illustrated in FIG. 11 is substantially similar to the implantable infusion device 100 described above with reference to FIGS. 1-4 and similar elements are represented by similar reference numerals. In addition to the elements associated with the implantable infusion device 100, implantable infusion device 100c includes a piezoelectric ultrasonic transducer 234 within the header assembly 142c. The catheter 122 is carried by the header assembly 142c such that the vibrations associated with the ultrasonic transducer 234 will propagate down the catheter and break up encapsulations and other deposits at the catheter outlet 124.

Another exemplary implantable infusion device that employs a piezoelectric actuator is generally represented by reference numeral 300 in FIGS. 12 and 13. The implantable infusion device 300 is similar to the implantable infusion device 100 in many respects and similar elements are represented by similar reference numerals. To that end, the exemplary infusion device 300 includes a housing 302 (e.g. a titanium housing) with a bottom portion 304, an internal wall 306, and a cover 308. An infusible substance (e.g. medication) may be stored in a reservoir 310 that is located within the housing bottom portion 304. The reservoir 310 may be replenished by way of a refill port 312 that extends from the reservoir, through the internal wall 306, to the cover 308. The reservoir 310 in the exemplary infusion device 300 is a positive pressure reservoir and, in the illustrated embodiment, the reservoir is in the form of a titanium bellows that is positioned within a sealed volume defined by the housing bottom portion 304 and internal wall 306. The remainder of the sealed volume is occupied by a propellant P that exerts a positive pressure on the bellows.

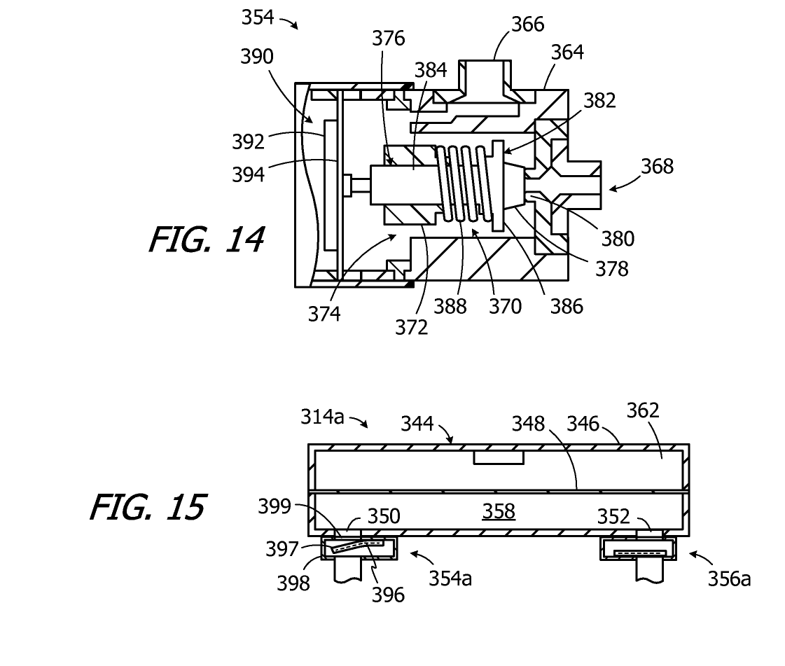

The exemplary infusion device 300 also includes a fluid transfer device 314 that is configured for use in combination with a positive pressure reservoir such as the exemplary positive pressure reservoir 310. In the illustrated embodiment, the fluid transfer device 314 has an accumulator 344 that includes a housing 346, a diaphragm 348 (e.g. a flexible sheet of titanium), an inlet 350, and an outlet 352. The fluid transfer device 314 also has an active inlet valve 354, which controls the flow of fluid into the housing inlet 350, and an active outlet valve 356, which controls the flow of fluid out of the housing outlet 352. The active inlet valve 354 is also connected to the interior of the positive pressure reservoir 310, while the active outlet valve 356 is also connected to the outlet port 318 which, in turn, may be connected to the catheter 322. The exemplary active valves 354 and 356 are discussed in greater detail below with reference to FIG. 14.

During operation of the fluid transfer device 314, infusible substance will move from the positive pressure reservoir 310 to an accumulator cavity 358, which is defined by the housing 346 and the diaphragm 348, when the active inlet valve 354 is opened. A pressure chamber 362 is located on the other side of the diaphragm 348. The active outlet valve 356 will be closed while the inlet valve 354 is opened. The diaphragm 348 will flex due to the positive pressure from the reservoir until it reaches a stop 360, as is shown in dashed lines in FIG. 13, thereby increasing the volume of the accumulator cavity 358 by a predetermined amount. The active inlet valve 354 will then be allowed to close. When the active outlet valve 356 is opened, the pressure within the chamber 362 will drive the diaphragm 348 back to the solid line position, thereby driving the predetermined volume of fluid to the outlet port 318.

Although the present fluid transfer device 314 is not so limited, the active inlet and outlet valves 354 and 356 in the illustrated embodiment are identical piezoelectrically-actuated valves. Turning to FIG. 14, the exemplary active inlet valve 354 (and outlet valve 356) includes a generally solid, cylindrical housing 364 with various open regions that accommodate portions of various structures and define a fluid flow path. More specifically, the housing 364 includes an inlet 366, an outlet 368 and an open region 370. The inlet 366 may be used as an outlet, and the outlet 368 may be used as an inlet, when the direction of fluid flow through the valve 354 is reversed. A spring retainer 372, with apertures 374 to permit fluid flow and a bore 376, is mounted within the housing 364. An elastomeric valve element 378 is movable in to and out of engagement with a rigid valve seat 380 that is associated with the outlet 368. The elastomeric valve element 378 is supported on a valve element retainer 382 that includes a shaft 384 and a spring retainer 386. A spring 388 (e.g. a coil spring), which is mounted between the spring retainers 372 and 386, biases the valve element retainer 382 to the closed position illustrated in FIG. 14 where the valve element 378 engages the valve seat 380.

With respect to actuation, the exemplary valve 354 in the fluid transfer device 314 has a piezoelectric actuator 390 that opens the valve. The actuator 390 includes a piezoceramic disk 392 that is carried by a flexible diaphragm 394 and may be connected to the valve element retainer 382 in the manner shown. When a voltage is applied across the piezoceramic disk 392, the disk will bend away from the valve seat 380 (i.e., to the left in FIG. 14) with a force sufficient to overcome the biasing force of the spring 388 and move the valve element 378 away from the valve seat 380 to open the active valve 354. The spring 388 will return the valve to the closed state when the voltage is removed. In other implementations, the flexible diaphragm may be omitted and the piezoelectric may consist of a stack of piezoceramic disks and/or a single piezoceramic disk that is fixedly supported at its outer periphery.

With respect to manufacturing and materials, the exemplary housing 364 may be a machined part and suitable materials for the housing include, but are not limited to, titanium, titanium alloys, stainless steel (e.g. 316L stainless steel), cobalt-nickel alloys, and refractory metals such as tantalum. The valve element retainer 382 may also be machined and suitable materials for the machined valve element include, but are not limited to, those described above in the context of the housing 364. Alternatively, the valve element retainer 382 may be molded. Suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve element 378 include silicone rubber, latex rubber, fluoropolymers, urethane, butyl rubber, and isoprene. The exemplary piezoceramic disk 392 may be formed from any suitable heat sterilizable piezoelectric ceramic including, but not limited to, the heat sterilizable piezoelectric ceramics described above.

Energy for the active valves 354 and 356, as well for other aspects of the exemplary infusion device 300, is provided by the implantable infusion device battery (not shown). The battery charges one or more capacitors in the manner described above, and is not directly connected to the active valves themselves. The capacitor(s) are selectively connected a piezoceramic disk 392, and disconnected from the battery, when a valve is opened, and are disconnected from the piezoceramic disks and connected to the battery when the valves are closed. As discussed above in the context of infusion device 100, the capacitor(s) are carried on a board along with an RF communication device that is connected to an antenna. The communication device may, alternatively, be an oscillating magnetic field communication device, a static magnetic field communication device, an optical communication device, an ultrasound communication device, a direct electrical communication device, or other suitable device. A controller 334 (FIG. 13), such as a microprocessor, microcontroller or other control circuitry, is carried on the other side of the board. The controller controls the operations of the infusion device 300 in accordance with instructions stored in memory and/or provided by and external device by way of the aforementioned communication device. For example, the controller 334 may be used to control the fluid transfer device 314 to supply fluid to the patient in accordance with, for example, a stored basal delivery schedule or a bolus delivery request, by selectively actuating (i.e., opening) and de-actuating (i.e., closing) the active valves 354 and 356.

Referring to FIGS. 12 and 13, the exemplary infusion device 300 is also provided with a side port 340 that is connected to a passageway between the outlet of the active valve 356 and the outlet port 318. The outlet port 318, a portion of the associated passageway, the antenna and the side port 340 may be carried by, for example, a molded plastic header assembly 342 that is secured to the housing 302. The housing 302 also includes a small aperture through which portions of the passageway are connected to one another, and a small aperture through which the antenna is connected to the board. The exemplary infusion device 300 may include a pressure sensor 345 between the active valve 356 and the outlet port 318 that may be employed in the manner described above with reference to pressure sensor 144.

Another exemplary piezoelectrically-actuated fluid transfer device is generally represented by reference numeral 314a in FIG. 15. The exemplary fluid transfer device 314a is substantially similar to fluid transfer device 314 and similar elements are represented by similar reference numerals. For example, the fluid transfer device 314a includes an accumulator 344 and active inlet and outlet valves. Here, however, the active inlet and outlet valves 354a and 356a are each formed primarily by a piezoelectric element 396 within a resilient elastomeric valve element 397. The active inlet and outlet valves 354a and 356a are identical but for the orientations of the piezoelectric element 396 and valve element 397. The elastomeric valve elements 397 are also located within housings 398 that define the valve seat and have lumens 399 which are aligned with the accumulator inlet 350 and outlet 352. The exemplary piezoelectric elements 396 may be formed from any suitable heat sterilizable piezoelectric ceramic including, but not limited to, the heat sterilizable piezoelectric ceramics described above.

The exemplary elastomeric valve elements 397 illustrated in FIG. 15 are biased to closed position (note outlet valve 256a). When a voltage is applied across one of the piezoelectric elements 396, the piezoelectric element will overcome the biasing force of the associated valve element 397 and move the valve element to the open position (note outlet valve 254a). The biasing force of the valve element 397 will return the valve element to the closed state when the voltage is removed.

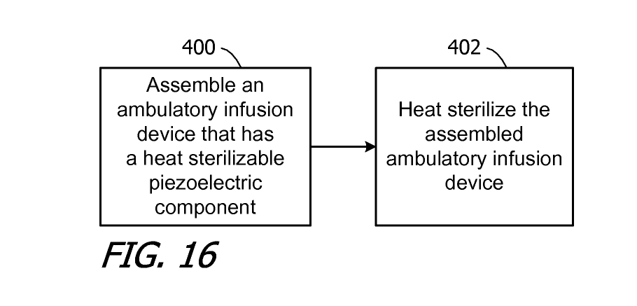

Turning to FIG. 16, one method of manufacturing an ambulatory infusion device includes the steps of assembling an ambulatory infusion device that has at least one component formed from a heat sterilizable piezoelectric material (Step 400) and heat sterilizing the assembled ambulatory infusion device with the component formed from a heat sterilizable piezoelectric material (Step 402). As noted above, the component formed from a heat sterilizable piezoelectric material (e.g. a heat sterilizable piezoceramic) may, for example, be associated with an alarm or a fluid transfer device. The heat sterilization process may, for example, be steam sterilization at temperatures of 121-132° C. for up to three hours, or may be dry heat sterilization at temperatures of 160-170° C. for up to two and one-half hours.

Although the inventions disclosed herein have been described in terms of the preferred embodiments above, numerous modifications and/or additions to the above-described preferred embodiments would be readily apparent to one skilled in the art. It is intended that the scope of the present inventions extend to all such modifications and/or additions and that the scope of the present inventions is limited solely by the claims set forth below.

We claim:

1. An ambulatory infusion device, comprising:
an external housing;
a reservoir within the external housing;
a fluid transfer device within the external housing and operably connected to the reservoir; and
an alarm including a heat sterilizable actuator within the external housing.

2. An ambulatory infusion device as claimed in claim 1, wherein the alarm comprises an audible alarm.

3. An ambulatory infusion device as claimed in claim 2, wherein the heat sterilizable actuator comprises a heat sterilizable piezoelectric actuator.

4. An ambulatory infusion device as claimed in claim 3, wherein the heat sterilizable piezoelectric actuator comprises a heat sterilizable piezoceramic actuator.

5. An ambulatory infusion device as claimed in claim 3, wherein the heat sterilizable piezoelectric actuator is secured to a portion of the external housing such that, when operating, the heat sterilizable piezoelectric actuator flexes the portion of the external housing inwardly and outwardly to produce sound waves.

6. An ambulatory infusion device as claimed in claim 1, wherein the alarm comprises a vibrating alarm.

7. An ambulatory infusion device as claimed in claim 6, wherein the vibrating alarm includes a heat sterilizable motor with a spindle and an imbalanced load carried by the spindle.

8. An ambulatory infusion device, comprising:
an external housing;
a reservoir within the external housing; and
a fluid transfer device, within the external housing, operably connected to the reservoir and including a heat sterilizable piezoelectric actuator.

9. An ambulatory infusion device as claimed in claim 8, wherein the heat sterilizable piezoelectric actuator comprises a flexible diaphragm and at least one piezoelectric element.

10. An ambulatory infusion device as claimed in claim 9, wherein the fluid transfer device comprises first and second fluid transfer devices and the heat sterilizable piezoelectric actuator is associated with both of the first and second fluid transfer devices.

11. An ambulatory infusion device as claimed in claim 10, wherein the first and second fluid transfer devices respectively include first and second housings and first and second pistons mounted for reciprocal movement within the first and second housings.

12. An ambulatory infusion device as claimed in claim 8, wherein the fluid transfer device includes a diaphragm and the heat sterilizable piezoelectric actuator is carried by the diaphragm.

13. An ambulatory infusion device as claimed in claim 12, wherein the diaphragm is carried by a valve plate such that a pump chamber is defined between the diaphragm and the valve plate.

14. An ambulatory infusion device as claimed in claim 8, wherein the heat sterilizable piezoelectric actuator comprises a heat sterilizable piezoceramic actuator.

15. An ambulatory infusion device as claimed in claim 8, wherein
the fluid transfer device includes an accumulator and first and second active valves; and
the heat sterilizable piezoelectric actuator is associated with at least one of the first and second active valves.

16. A method, comprising the step of:
heat sterilizing an ambulatory infusion device that includes at least one component formed from a heat sterilizable piezoelectric material.

17. A method as claimed in claim 16, further comprising the step of:
assembling the ambulatory infusion device prior the heat sterilizing step.

18. A method as claimed in claim 17, wherein the step of assembling comprises assembling an ambulatory infusion device that includes a piezoelectric alarm prior the heat sterilizing step.

19. A method as claimed in claim 17, wherein the step of assembling comprises assembling an ambulatory infusion device that includes a piezoelectric fluid transfer device actuator prior the heat sterilizing step.

20. A method as claimed in claim 16, wherein heat sterilizing comprises steam sterilizing an ambulatory infusion device, that includes at least one component formed from a heat sterilizable piezoelectric material, at a temperature of about 121-170° C.
